(19) United States
(12) Patent Application Publication
(10) Pub. No.: US 2003/0069581 A1
(43) Pub. Date: Apr. 10, 2003

(54) Universal intramedullary nails, systems and methods of use thereof

(76) Inventors: David T. Stinson, Woodinville, WA (US); R. Trigg McClellan, Nashville, TN (US)

Correspondence Address: BOZICEVIC, FIELD & FRANCIS LLP, 200 MIDDLEFIELD RD, SUITE 200, MENLO PARK, CA, 94025, US

(21) Appl. No.: 09/971,445

(22) Filed: Oct. 4, 2001

(51) Int. Cl. A61B 17/58

(52) U.S. Cl. 606/62

(57) ABSTRACT

Devices, systems and methods are provided for treating fractures and correcting malunions and non-unions of long bones. The subject devices include intramedullary nails for implantation into a long bone. The intramedullary nails are configured such that they are very versatile and usable for treating many types of fractures, malunions and non-unions in both left and right corresponding long bones and by means of either an antegrade or retrograde approach. In particular, the subject nails provide a universal bore pattern for receiving one or more screws for locking the nail within the bone. The subject systems include ancillary and accessory devices for implantation of the intramedullary nails. The methods of the present invention include using the subject intramedullary nails to treat many fractures, malunions and non-unions in both left and right corresponding long bones and by means of either an antegrade or retrograde approach, and employing the subject systems to insert and secure the subject nails within such long bones.

### FIELD OF THE INVENTION

The present invention relates to orthopedic surgery, and more particularly to surgical implants for the intramedullary treatment of long bone fractures and the correction of bone malunions and non-unions.

### BACKGROUND OF THE INVENTION

The use of intramedullary nails or rods has become the gold standard for the treatment of fractures, malunions and non-unions of long bones, i.e., the femur and tibia in the lower extremities. The typical surgical procedure for such treatment involves the insertion or implantation of a nail or rod into the intramedullary canal of the subject bones such that the nail spans the fracture(s) and/or non-union or malunion. Interlocking screws are then placed through bores or apertures within the intramedullary nail, interlocking with the nail and extending through the bone on both sides of the treatment site. As such, the bone and/or bone fragments are stabilized and immobilized against rotational and lateral movement in order to allow the bone to properly set and heal, and to prevent displacement of the fracture during the healing process.

Because of the wide range of long bone lengths and diameters, it is necessary for a surgeon or hospital to stock at least a base quantity of intramedullary nails to treat a diverse patient population and injury patterns. As such, hospitals typically stock a selection of intramedullary nails for each left and right long bone having lengths and corresponding diameters within practical ranges. Such a base inventory of nails may include hundreds of nails, which can be very costly.

The popularity of intramedullary nails has lead to technological advances in the area of long bone repair or reconstructions, including the development of highly specialized nail designs to address a variety of indications. However, this has lead to the need to stock an even greater inventory of nails as well as their ancillary components further increasing the cost burden on hospitals and third party payers.

Attempts have been made to address the staggering inventory requirements of hospitals by modularizing intramedullary nails, related instrumentation and kits. For example, certain intramedullary design changes have alleviated the need to stock left and right corresponding nails for each bone. Such modular intramedullary nail systems require the surgeon to assemble an intramedullary nail having the desired specifications from a selection of nail base portions to accommodate either a left or right bone. While this modular system reduces the total number of nails in inventory, the actual inventory is only marginally reduced due to the need to now stock left and right portions for the range of diameters.

Other available modular intramedullary nail systems aim at reducing inventory by providing a selection of interchangeable sleeves which fit over a generic nail to customize the nail for application to different types of fracture patterns or indications. One such intramedullary system for treatment of the femur is disclosed in U.S. Pat. No. 6,120,504. This system provides a nail having a slot along the length of its proximal portion or end and three interchangeable sleeves each having one or two transverse bores oriented in a particular pattern for addressing three groups of fractures, e.g., those requiring interlocking fixation, those requiring reconstructive fixation and those requiring retrograde fixation. This system adds complexity to the system requiring “assembly” of the nail and increases the potential for insertion of the wrong implant. There is also potential for implant failure at the interface of these modular components. Finally, use of this system increases the surgical time required for “assembly”. Again, while this system also reduces the total number of nails in inventory, the reduction is somewhat marginal as the system only addresses fractures to the femur.

There are still other disadvantages of modular nail systems. Such systems increase the number and complexity of particular techniques available to a surgeon, requiring the need for increased surgeon training, a costly endeavor which may negate any cost savings in inventory reductions. This complexity may also increase the risk of selecting an improper nail for implantation and/or improperly implanting a nail. Additionally, such modular components are more susceptible to biomechanical issues and are more likely to experience failure when placed under stress and strain.

Another currently available intramedullary nail system is the Stryker® Howmedica Osteonics Antegrade/Retrograde Compression Femoral Nailing System which attempts to overcome some of the disadvantages of modular systems. This system has the advantage of reducing the necessary inventory of nails for treatment of the femur by providing a single intramedullary nail design for the treatment of the most common types of fractures in the femur by means of either an antegrade or retrograde approach. Additionally, this system reduces the necessary inventory of nails for treatment of the tibia by providing another intramedullary nail design for treatment of the most common types of fractures in the tibia. While an improvement to other prior art systems, the Howmedica nails are limited to either femoral or tibial applications only (not both) and, thus, the femoral and tibial nails, respectively, require a different set of ancillary components for inserting and locking the nail in position within the target long bone.

Thus, there is still a need for an intramedullary nail and system which will further reduce the inventory costs to the hospital by reducing the number of intramedullary nails and ancillary components that are necessary to be inventoried, increase the ease and simplicity of intramedullary insertion procedures, reduce the risk of improper nail selection and insertion, and address the majority of fractures in more than one kind of long bone.

### SUMMARY OF THE INVENTION

Devices, systems and methods are provided for treating fractures and correcting malunions and non-unions of long bones. The subject invention includes intramedullary nails for implantation into a long bone and which are configured such that they are very versatile and usable for treating many types of fractures, malunions and non-unions in both left and right corresponding long bones and by means of either an antegrade or retrograde approach. In particular, the subject nails provide a universal bore pattern, also referred to as an interlocking bore pattern, of screw bores for receiving one or more interlocking bone screws for locking the nail within the bone. Because the selected bore pattern is common to both the antegrade femoral, retrograde femoral and tibial nails thereby allowing a single instrument guide for the insertion of the locking screws.

Each of the subject intramedullary nails defines a longitudinal reference plane wherein the longitudinal axis of each screw bore is positioned in the range from about negative 60° to about positive 60° with respect to the longitudinal reference plane. Each of the nails further defines a transverse reference plane perpendicular to the longitudinal reference plane wherein the longitudinal axis of each screw bore is positioned in the range from about negative 60° to about positive 60° with respect to the transverse reference plane.

The systems of the present invention may include ancillary and accessory devices for implantation of the intramedullary nails. Certain subject systems include at least one intramedullary nail of the present invention having a first universal bore pattern having a plurality of screw bores for receiving screws wherein each screw bore defines a longitudinal axis. These systems further include a guide member having a second universal bore pattern having a plurality of guide bores for aligning and guiding the bone screws to be received into the plurality of screw bores. Each guide bore defines a longitudinal axis which is coaxial with the longitudinal axis of a corresponding screw bore. The ratio of screw bores to guide bores is preferably 1 to 1.

Certain other subject systems include at least one intramedullary nail configured for treating the femur and at least one intramedullary nail configured for treating the tibia. Each femoral nail and each tibial nail of the system includes a plurality of screw bores at its proximal end for receiving bone screws wherein each plurality of screw bores defines a selected screw bore pattern universal to all nails within the system. As such, only a single configuration of bone insertion and locking instrumentation is needed for all of the nails. Moreover, the at least one femoral nail is configured for antegrade and retrograde implantation in a left and right femur and the tibial nail is configured for antegrade implantation in a left and right tibia.

In the methods of the present invention, the subject intramedullary nails are used to treat fractures, malunions and non-unions in both left and right corresponding long bones and by means of either an antegrade or retrograde approach. One exemplary method of the present invention for implanting an intramedullary nail into a long bone includes the following general steps: providing at least one or a plurality of intramedullary nails of the present invention having a universal bore pattern, as described above, and a screw guide member having a bore pattern corresponding to the bore pattern of the nail; selecting an appropriate intramedullary nail for the application (taking into consideration the type of long bone, the type of fracture, the size of the bone, etc); forming a nail entry site at an appropriate location proximate the targeted long bone; through the entry site, preparing the medullary canal of the long bone for receiving the intramedullary nail; inserting the intramedullary nail into the prepared medullary canal; determining which of the screw bores of the intramedullary nail to use as fixation points within the bone; operatively aligning the guide bores of said guide member with the screw bores of the intramedullary nail, wherein an alignment path is defined between each guide bore and each screw bore (i.e., the longitudinal axis of a screw bore is co-axially aligned with the longitudinal axis of the guide bore); drilling a screw hole within the bone at each fixation point by means of a drill bit guided along the alignment path; and delivering a bone screw into each screw hole.

The subject methods may be employed with any suitable long bone, and are particularly suitable for implanting the subject nails within a femur by means of either an antegrade or retrograde approach and within a tibia by means of an antegrade approach. Where a femur is the target bone and the desired approach is antegrade, the entry site is made adjacent to the proximal end of the femur. However, where the desired approach is retrograde, the entry site is made adjacent to the distal end of the femur. Where a tibia is the target bone and the desired approach is antegrade, the entry site is made adjacent to the proximal end of the tibia.

Also provided by the present invention are kits for treating long bones. Certain subject kits include a plurality of intramedullary nails of the present invention having various length and diameter dimensions wherein each intramedullary nail has the same screw bore pattern. Certain of these nails are configured for treating both left and right femurs, for example, and other nails are configured for treating both left and right tibias. Furthermore, each femoral nail may be configured for implantation by an antegrade approach and a retrograde approach, and each tibial nail may be configured for implantation by an antegrade approach.

These and other objects, aspects, advantages and features of the invention will become apparent to those skilled in the art upon reading this disclosure in combination with the accompanying figures.

### BRIEF DESCRIPTIONS OF THE DRAWINGS

FIG. 1 illustrates an embodiment of the intramedullary nail of the present invention particularly useful for treating femoral fractures, malunions and non-unions.

FIG. 2 illustrates an embodiment of the intramedullary nail of the present invention particularly useful for treating tibial fractures, malunions and non-unions.

FIG. 3 illustrates an enlarged view of a proximal end portion of the intramedullary nails of FIGS. 1 and 2 having a universal or common proximal bore pattern.

FIG. 4A illustrates a frontal or coronal view of a human femur operatively engaged at its proximal end with certain components of a nail insertion system of the present invention for inserting the intramedullary nails of the present invention into the medullary canal of the femur. FIG. 4B is a lateral or sagital view of the femur and the engaged components of FIG. 4A.

FIG. 5A illustrates a frontal or coronal view of a human femur operatively engaged at its proximal end with certain components of a nail insertion system of the present invention for drilling a selection of bone screws into the femur in order to secure the intramedullary nail of FIG. 1 within an open channel created within the femur. FIG. 5B is a lateral or sagital view of the femur and the engaged components of FIG. 5A.

FIG. 6A illustrates a frontal or coronal view of a human femur having the intramedullary nail of FIG. 1 (shown in phantom) implanted therein in an antegrade direction. FIG. 6B is a lateral or sagital view of the femur and the implanted intramedullary nail (shown in phantom) of FIG. 6A.

FIG. 7A illustrates a frontal or coronal view of a human femur operatively engaged at its distal end with certain components of a nail insertion system of the present invention for inserting the intramedullary nail of FIG. 1 within an open channel created within the femur. FIG. 7B is a lateral or sagital view of the femur and engaged components of FIG. 7A.

FIG. 8A illustrates a frontal or coronal view of a human femur operatively engaged at its distal end with certain components of a nail insertion system of the present invention for drilling a selection of bone screws into the distal end of the femur in order to secure the intramedullary nail of FIG. 1 within an open channel created within the femur. FIG. 8B is a lateral or sagital view of the femur and engaged components of FIG. 8A.

FIG. 9A illustrates a frontal or coronal view of a human femur having the intramedullary nail of FIG. 1 (shown in phantom) implanted therein in a retrograde direction. FIG. 9B is a lateral or sagital view of the femur and implanted intramedullary nail (shown in phantom) of FIG. 9A.

FIG. 10A illustrates a frontal or coronal view of a human tibia operatively engaged at its proximal end with certain components of a nail insertion system of the present invention for inserting the intramedullary nail of FIG. 2 into an open channel created within the intramedullary canal of the tibia. FIG. 10B is a lateral or sagital view of the tibia and engaged components of FIG. 10A.

FIG. 11A illustrates a frontal or coronal view of a human tibia operatively engaged at its proximal end with certain components of a nail insertion system of the present invention for drilling a selection of bone screws into the tibia for securing the intramedullary nail of FIG. 2 within an open channel created within the tibia. FIG. 11B is a lateral or sagital view of the tibia and engaged components of FIG. 11A.

FIG. 12A illustrates a human tibia having the intramedullary nail of FIG. 2 (shown in phantom) implanted therein in an antegrade direction. FIG. 12B is a lateral or sagital view of the tibia and the implanted intramedullary nail (shown in phantom) of FIG. 12A.

FIG. 13A illustrates an enlarged view of a proximal end portion of the intramedullary nails of the present invention having another embodiment of a universal proximal bore pattern of the present invention. FIGS. 13B and 13C are cross-sectional views of the proximal end portion of the intramedullary nail of FIG. 13A taken along lines B-B and C-C, respectively.

FIG. 14A illustrates certain components of a nail insertion system of the present invention for drilling a selection of bone screws into a long bone. FIG. 14B illustrates a bottom view of the nail insertion system of FIG. 14A. FIG. 14C illustrates a cross-sectional view of a portion of the nail insertion system taken along line C-C of FIG. 14B.

### DETAILED DESCRIPTION OF THE INVENTION

Devices, systems and methods are provided for treating fractures and correcting malunions and non-unions of long bones. The subject devices include intramedullary nails for implantation into a long bone. The subject systems include ancillary and accessory devices for implantation of the intramedullary nails. The intramedullary nails are configured such that they are very versatile and usable for treating many types of fractures, malunions and non-unions in both left and right corresponding long bones. In particular, the subject nails provide a universal or common bore pattern, also referred to as a universal interlocking bore pattern, for receiving one or more interlocking bone screws for locking the nail within the bone. Another feature of the present invention is a device or assembly of components for implanting the intramedullary nails of the present invention into the target long bone. In particular, an implantation device or system is provided having a single configuration applicable for all nails of the present invention having the same universal bore pattern and, in particular, provides a drill guide having a universal drill guide pattern corresponding to the universal proximal bore pattern of the intramedullary nails of the present invention. As such, the implantation device is also universal, applicable to insertion and drilling of bone screws into the subject nails for treating or correcting various defects or injuries of the left and right sides of various long bones. One method of the present invention involves using the subject intramedullary nails to treat many fractures, malunions and non-unions in both left and right corresponding long bones and, in the femur, by means of either an antegrade or retrograde approach. Another method of the present invention involves using the subject implantation devices and system to implant and affix the subject nails into various long bones to treat many fractures and malalignment locations in both left and right corresponding long bones and, in most cases, by means of either an antegrade or retrograde approach.

Before the subject devices, systems, methods and kits are described, it is to be understood that this invention is not limited to the particular embodiments described and illustrated, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a”, “and”, and “the” include plural referents unless the context clearly dictates otherwise.

All publications mentioned herein are incorporated herein by reference to disclose and describe the methods and/or materials in connection with which the publications are cited. These publications are provided solely for their disclosure prior to the filing date of the present application. Nothing herein is to be construed as an admission that the present invention is not entitled to antedate such publication by virtue of prior invention. Further, the indicated publication dates of the respective publications may be different from the actual publication dates which may need to be independently confirmed.

Definitions

The term “antegrade” as used herein means the approach or fashion by which an intramedullary nail is implanted or inserted into the medullary canal of a long bone. An antegrade approach with respect to the long bones of the lower extremities, involves inserting the intramedullary nail from the end of the bone which is anatomically closer to the hip (i.e., furthest from the foot). In the context of treating the femur, the intramedullary nail is inserted into the proximal end of the femur at the piriformis fossa. In the context of treating the tibia, the intramedullary nail is inserted into the proximal end of the tibia. An antegrade approach with respect to the long bones of the arm involves inserting the intramedullary nail from the end of the bone that is anatomically closer to the shoulder (i.e., furthest from the hand). In the context of treating the humerus, the intramedullary nail is inserted into the proximal end of the humerus.

The term “retrograde” as used herein means the approach or fashion by which an intramedullary nail is implanted or inserted into the medullary canal of a long bone. A retrograde approach, with respect to the long bones of the lower extremities, involves inserting the intramedullary nail from the end of the bone that is anatomically furthest from the hip (i.e., closer to the foot). In the context of treating the femur, the intramedullary nail is inserted into the distal end of the femur. A retrograde approach, with respect to the long bones of the arm, involves inserting the intramedullary nail from the end of the bone that is anatomically closest to the hand (i.e., furthest from the shoulder). In the context of treating the humerus, the intramedullary nail is inserted into the distal end of the humerus.

The term “proximal end” or “proximal end portion” as used herein with respect to the intramedullary nails of the present invention means the end or end portion of the intramedullary nail providing a universal interlocking bore pattern of the present invention, regardless of whether the intramedullary nail is inserted into the bone in an antegrade or retrograde fashion.

The term “distal end” or “distal end portion” as used herein with respect to the intramedullary nails of the present invention means the end or end portion of the intramedullary nail opposite the proximal end or proximal end portion of the intramedullary nail.

Devices

As summarized above, the invention provides devices, including intramedullary nails, for treating a variety of fractures, malunions and non-unions of the long bones. With respect to the following discussion, the structure and application of the subject devices will be primarily described in the context of treating and correcting the femur and tibia; however, such is intended to be exemplary and is not in any way intended to limit the scope of the invention.

The subject intramedullary nails provide a universal bore pattern for receiving one or more interlocking screws or pins for locking the intramedullary nail once the nail is selectively positioned within the medullary canal of the bone being treated. This universal bore pattern provides a selected number of bores, wherein the bores may have the same diameter (from bore to bore and from universal bore pattern to universal bore pattern) and are selectively located along the length of and substantially transverse (i.e., either perpendicular to or canted or anteverted within a limited range) to the intramedullary nail or to the proximal end portion thereof. The subject universal bore patterns may alternatively be provided on a modular sleeve or extension which is attachable to a nail base portion.

For purposes of describing the present invention, the universal bore patterns and their respective bores will be described and referenced with respect to two selected reference planes, a longitudinal reference plane and a transverse reference plane, associated with the intramedullary nail or a portion thereof, but it is understood that any appropriate reference planes, lines or points may be used to describe the relative positioning of the bores of the universal bore patterns of the present invention.

In any embodiment, each bore defines a longitudinal axis relatively positioned at selected angles, at least in part, with respect to the longitudinal axis of the nail, or a portion thereof and, thus, a “longitudinal reference plane” which includes such longitudinal axis. Additionally, the longitudinal axis of each bore is relatively positioned at selected angles with respect to another axis or plane which is perpendicular or transverse to the nail's (or portion thereof) longitudinal axis, referred to as the “transverse reference plane.” This transverse reference plane may be defined by another feature or aspect, e.g., an attachment or orientation groove, of the intramedullary nail. As such, the axis of each bore may lie parallel to or at an angle with one or more of the other bore axes, and may lie in the same plane with or different planes from one or more of the other bore axes. The specific parameters within which a particular bore pattern is universally applicable and within the scope of the present invention will be discussed in detail below.

Each interlocking bore pattern employed by an intramedullary nail of the present invention, or an attachable nail extension thereof, may be used to treat at least the left and right femur, the left and right tibia and the left and right humerus; however, the same such bore pattern may also be useful for treating the left and right of the other long bones of the upper and lower extremities. Additionally, each interlocking bore pattern of the present invention may be used with antegrade and retrograde approaches to treat the femur and humerus as well as with an antegrade approach to treat the tibia. As such, a bore pattern of the present invention is applicable in at least six different locations and positions, including at least the left femur in an antegrade configuration, the left femur in a retrograde configuration, the right femur in an antegrade configuration, the right femur in a retrograde configuration, the left tibia in an antegrade configuration and the right tibia in an antegrade configuration.

Furthermore, each subject bore pattern of the present invention may be used to treat a multiplicity of fractures, malunions or non-unions including, but not limited to, open and closed femoral shaft and tibial fractures, ipsilateral femoral neck and/or shaft fractures, supracondylar femoral fractures with or without intra-articular extension, impending pathological femoral and tibial fractures, fractures distal to a hip prosthesis, fractures proximal to a knee arthoplasty or knee implant, non-unions and malunions of the femur and of the tibia, pseudarthrosis of the femoral and tibial shafts, corrective oseotomies of the femur and tibia, transverse, oblique, comminuted and spiral fractures of the femur and tibia, metaphyseal fractures of the tibia, and the like. Thus, taking into consideration at least six suitable bone locations/positions suitable for treatment by the intramedullary nails of the present invention and the numerous applicable indications that can be treated by those nails, the permutations of these factors results in an exponential number of applications for which the subject intramedullary nails are suited.

The present invention further includes ancillary devices and components for implanting and securing the intramedullary nails of the present invention within a long bone. Such ancillary devices include but are not limited to a drill guide, drill bits, screw insertion sleeves, screw drive shafts, etc., lend themselves to a universal configuration. For example, only a single-configuration drill guide, having a universal interlocking screw guide pattern corresponding to that of the universal or common interlocking bore pattern of the intramedullary nail is necessary for use in guiding, drilling and securing the interlocking screws into the respective bores of the intramedullary nails or attachable nail extensions of the present invention. Additionally, as the present invention provides universal bore patterns having bores with the same diameter from bore to bore, as well as from bore pattern to bore pattern, one size bone screw may be used. Thus, in addition to drastically reducing the total number of intramedullary nails required to be stocked by a hospital, the present invention also serves to significantly reduce the number of ancillary components for implanting the intramedullary nails and/or attachable nail extensions of the present invention as well as the number of bone screws which are necessary to be inventoried.

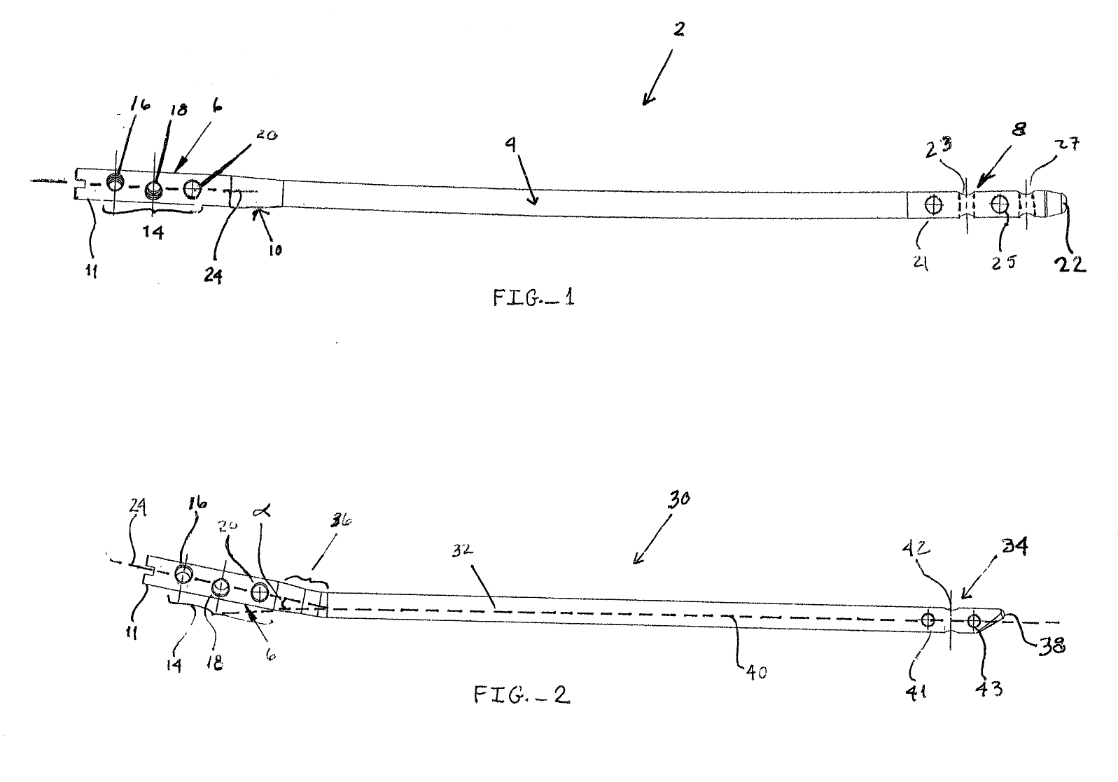

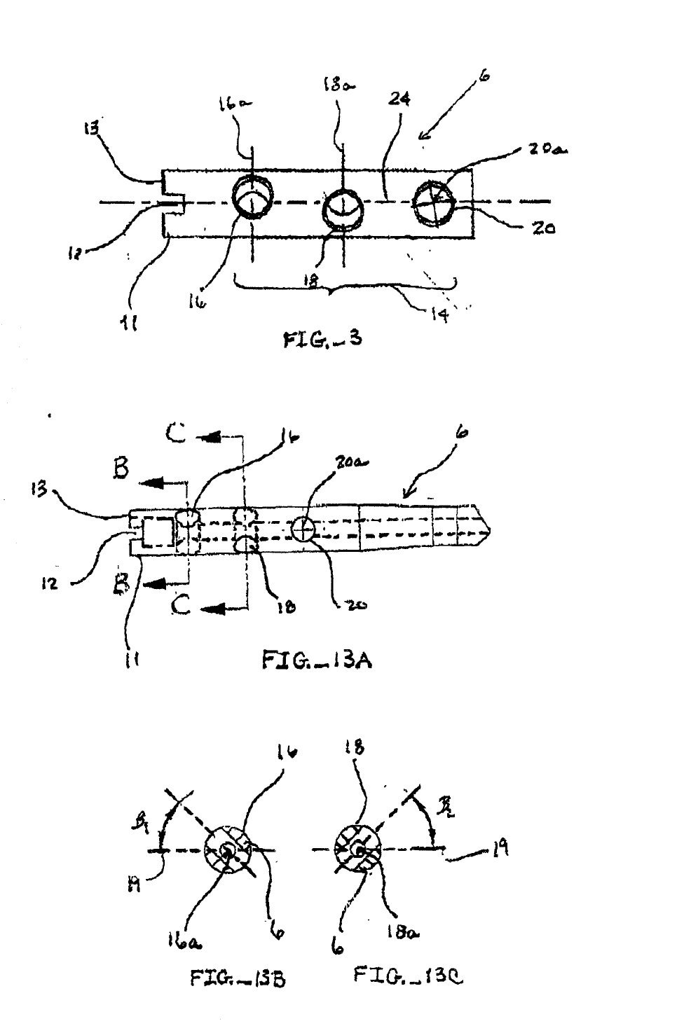

Referring now to the drawings, FIGS. 1 and 2 illustrate intramedullary nails of the present invention, each employing an embodiment of a universal or common bore pattern at a proximal end thereof. Intramedullary nail 2 of FIG. 1 is particularly configured for use in treating the femur, having a substantially straight configuration, and preferably having a slight curve (e.g., having a radius of curvature of about 3 to 5 meters and more typically about 4 to 4.2 meters, more or less) along its length. Intramedullary nail 2 includes, in general, proximal and distal end portions 6, 8 and a body portion 4 extending there between, collectively defining a longitudinal axis of nail 2. Situated between proximal end portion 6 and body portion 4 is a transition segment 10 having a diameter which tapers down from proximal end portion 6 to body portion 4. Extending from proximal end 11 and distal tip 22 of distal end portion 8 is a central, thru-cannula or lumen (not shown) for ease of passage of intramedullary nail over small diameter guide wire within medullary canal.

Distal end portion 8 of nail 2 has transverse distal locking bores 21, 23, 25 and 27 for receiving corresponding bone screws (not shown) for locking distal end portion 8 to a bone once operatively placed within the medullary canal. For example, bores 21 and 25 may be used for antegrade approaches for inserting nail 2, and bores 23 and 27 may be used for retrograde approaches for inserting nail 2, or visa-versa. Additionally, only one of the bores or more than two bores, in any pairing arrangement, may be employed, i.e., receive a bone screw. The longitudinal axes of distal bores 21 and 25 are preferably substantially parallel to each other, and substantially perpendicular to the longitudinal axis of bores 23 and 27, the latter of which are also substantially parallel to each other. Distal bores 21, 23, 25 and 27 may be either threaded or un-threaded and have diameters in the range from about 5.6 to 7 mm, and more typically from about 6.0 to 6.1 mm, and are preferably the same size. Distal bores 21, 23, 25 and 27are positioned approximately 50 mm, 37 mm, 25 mm and 15 mm, respectively, from distal tip 22. However, the distal bores may be spaced other suitable distances either farther from or closer to distal tip 22. Furthermore, the distal bores may be spaced any suitable distance from each other including being evenly spaced from each other. Although four distal bores are illustrated less than or more than four may also be provided, however, not all distal bores provided need be used and will depend on the type and location of the fracture being treated as well as surgeon preference. Further, such configuration of distal bores is only exemplary and other appropriate configurations are within the scope of the present invention. Distal tip 22 may have a slightly tapered configuration to facilitate ease of insertion within the bone.

In FIG. 2, intramedullary nail 30 is particularly configured for use in treating the tibia, having the same universal bore pattern as nail 2 of FIG. 1 in its proximal end portion 6, and a distal end portion 34 and a body portion 32 extending there between and having a substantially straight configuration and defining a longitudinal axis 40. Situated between proximal end portion 6 and body portion 32 is a transition segment or elbow 36, providing a slight bend for facilitating insertion into the tibia. The intersection of longitudinal axis 24 of proximal end portion 6 and longitudinal axis 40 of body portion 32 define an angle α, which generally ranges between about 0° and 20°, and more typically between about 9° and 12°. Extending from a proximal end 11 of proximal end portion 6 and distal tip 38 of distal end portion 34 is a central, thru-cannula or channel or lumen (not shown) for ease of passage of intramedullary nail over a small diameter guide wire within medullary canal.

Distal end portion 34 of nail 30 has transverse distal bores 41, 42 and 43 for receiving corresponding bone screws (not shown) for locking distal end portion 34 to the bone once operatively placed within the medullary canal. The longitudinal axes of bores 41 and 43 are preferably parallel to each other and perpendicular to the longitudinal axis of bore 42. Distal bores 41, 42 and 43 may be threaded or unthreaded and have diameters in the range from about 4.2 to 5 mm, and more typically from about 4.2 to 4.7 mm, and preferably have the same diameter from bore to bore and from distal bore pattern to distal bore pattern of the intramedullary nails of the present invention. Distal bores 41, 42 and 43 are positioned approximately 24 mm, 16 mm and 8 mm, respectively, from distal tip 38. However, the distal bores may be spaced farther from or closer to distal tip 22. Furthermore, the distal bores may be spaced any suitable distance from each other including being evenly spaced from each other. Although three bores are illustrated in such a configuration, one, two or more than three in any appropriate arrangement may also be used depending on the type and location of the fracture being treated as well as surgeon preference. Distal tip 38 may have a slanted or beveled configuration to facilitate ease of insertion within the bone.

While proximal end portion 6 has thus far been described as a contiguous, integrated component of a one-piece nail, proximal end portion 6 may itself be a modular extension which is interchangeable with other like or similar proximal end portions of the present invention and which is attachable to nails (e.g., nails having body and distal end portions) having various lengths and diameters. The attachment of proximal end portion 6 and a nail base may be accomplished by any appropriate means, such as a threaded screw configuration.

Referring now to FIG. 3, there is shown an enlarged illustration of proximal end portion 6 having universal transverse bore pattern 14 which, in this embodiment, includes, starting from proximal end 11, bores 16, 18, and 20 in a substantially linear arrangement along the length of proximal end portion 6. At least proximal end portion 6, and, in certain embodiments, the entire length of nail 2, defines a longitudinal axis 24. As mentioned above, for purposes of describing the invention and particularly the relative positioning of the bores of the universal proximal bore pattern 14, two reference planes are herein defined with respect to the longitudinal axis 24. Proximal end 11 of proximal end portion 6 defines a proximal surface 13 which defines a first reference plane, a “transverse reference plane,” which is perpendicular to longitudinal axis 24. Extending across the diameter of surface 13 is an attachment or orientation groove or notch 12 which provides a means by which an ancillary instrument, such as a tool for inserting and/or orienting the position of an intramedullary nail within the medullary canal, may be securely engaged with or attached to an intramedullary nail 2. The length of groove 12 defines a groove axis (not referenced in FIG. 1) which is parallel to and/or lying within the proximal end plane and which is perpendicular to longitudinal axis 24. The intersection of this groove axis and longitudinal axis 24 defines a second reference plane, a “longitudinal reference plane,” which is parallel to the proximal end plane. The transverse and longitudinal reference planes have been defined herein for the purpose of facilitating the description of the relative positions of the proximal bores of the universal bore patterns of the present invention. It should be noted, and understood by those skilled in the art, that any other reference points, lines, planes or structures, imaginary or real, of the intramedullary nails of the present invention may be used to define the relative positioning of the bores.

Each proximal bore 16, 18 and 20 defines a longitudinal axis 16a, 18a, 20a, respectively, having a relative position definable with respect to both the transverse and longitudinal reference planes, defined above. First proximal bore 16 has a longitudinal axis 16a which is approximately 15 mm from or distal of surface 13, and which is parallel to the proximal end plane and canted a positive 15°, for example, about the longitudinal reference plane (i.e., from the perspective of FIG. 1). Second proximal bore 18 has a longitudinal axis 18a which is approximately 30 mm from or distal of surface 13, and which is parallel to the transverse reference plane and canted a negative 15°, for example, about the longitudinal reference plane (i.e., from the perspective of FIG. 1). Third proximal bore 20 has a longitudinal axis 20a which is approximately 45 mm from distal surface 13, and which is parallel with the transverse reference plane (and the groove axis) and lies within the longitudinal reference plane, i.e., is perpendicular to and intersects longitudinal axis 24. In the particular bore pattern embodiment illustrated in FIG. 3, proximal bore 18 is approximately equally spaced between proximal bore 16 and proximal bore 20, and longitudinal axis 20a of proximal bore 20 approximately bisects the angular distance between longitudinal axes 16a and 18a of proximal bores 16 and 18, respectively. However, the proximal locking bores may be spaced apart any appropriate distance and may extend from the proximal surface 13 any appropriate distance.

The universal proximal bore pattern 14 of the nails of FIGS. 1, 2 and 3 are only exemplary of the present invention. Generally, the proximal bore patterns of the present invention have at least three bores; however, not all of the bores are required to be used (i.e., receive a bone screw) in a single application to fulfill the purposes of the present inventions. In other words, certain applications will require that all three bores be employed while others will require only one to two of the proximal bores to be employed. Many factors, including but not limited to the particular bone being treated, the insertion approach (antegrade or retrograde) employed, the type and location of the fracture, malunions or non-unions, as well as surgeon preference, will dictate how many and which bores (and corresponding bone screws) are actually used. For example, in some device embodiments of the present invention used for treating certain femoral fractures, such as open or closed femoral shaft fractures, subtrochanteric fractures, nonunions and malunions, pseudarthroses and corrective osteotomies, in which an antegrade approach is used, only one bone screw, typically the most distal in the universal pattern may used and for treating certain femoral fractures, such as fractures distal to a hip prostheses, ipsilateral femoral neck/shaft fractures, open or closed femoral shaft fractures, supracondylar fractures, nonunions and malunions, pseudarthroses and corrective osteotomies, in which an retrograde approach is used, three bone screws in the universal pattern may used. For treating certain tibial fractures, such as transverse fractures, oblique and spiral fractures, segmental fractures, comminuted fractures, pseudarthroses and corrective osteotomies, nonunions and malunions, metaphyseal and diaphyseal fractures an antegrade approach is used, and depending on fracture location 1, 2 or 3 bone screws may bone screws may be used in the proximal end of the nail.

Moreover, the relative positioning of the respective bores with respect to each other and with respect to the proximal end portion may vary within certain parameters while still achieving the objectives of the present invention, e.g., providing a universal interlocking proximal bore pattern and corresponding nail insertion instrumentation for treating a variety of fractures in a variety of bone locations, thus reducing the necessary inventory of such components. Such parameters include, but are not limited to, the distance of each of the proximal bores from the proximal end of the nail, the relative distance between the bores, and the axial position of each bore relative to the intramedullary nail and to the other proximal bores.

The distance of each proximal bore from proximal end 11 may increase at a fixed increment, such as 15 mm increments as in the embodiments of FIGS. 1 and 2, or may increase at increments of varying lengths. As such, the spacing between adjacent bores may be fixed or vary throughout the bore pattern but, in either case, is in the range from about 8 to 75 mm, typically from about 15 to 60 mm and more typically from about 15 to 45 mm.

Furthermore, the relative positions of the longitudinal axes of the respective proximal bores of the universal proximal bore patterns of the present invention may vary with respect to the selected reference planes as well as with respect to each other. As illustrated in the embodiment of FIGS. 13A-C, generally, each of the proximal bores may be canted, anteverted or retroverted within the range from about positive 60° (as indicated by angle β1 in FIG. 13B) to about negative 60° (as indicated by angle β2 in FIG. 13C) from the longitudinal reference plane 19; provided however, the angle between the longitudinal axis of any two proximal bores is not greater than about 90°. Moreover, each of the proximal bores may be positioned so as to have its longitudinal axis canted, anteverted or retroverted towards or away from the transverse reference plane, in the range from about 125° to 150°.

Generally, the intramedullary nails of the present invention, in both integral and modular embodiments, may have proximal end, body and distal end portions having diameters, lengths and bore diameters which are the same or similar to those of prior art nails. As such, the nails of the present invention have body portion lengths in the range from about 26 to 48 cm for femoral nails and from about 25 to 42 cm for tibial nails, and have shorter lengths for the long bones of the upper extremities. The proximal end portions or proximal modular extensions of the intramedullary nails of the present invention have lengths in the range from about 7 to 9 cm and more typically from about 7.8 to 8.0 cm for femoral nails and from about 6 to 8 cm and more typically from about 6.2 to 7.0 cm for tibial nails. The distal end portions of the intramedullary nails of the present invention, measured from the distal tip 22 of the femoral nail to the distal bore furthest from distal tip 22, have lengths in the range from about 5 to 7 cm and more typically from about 5.0 to 5.5 cm for femoral nails and from about 2 to 5 cm and more typically from about 2.4 to 2.6 cm for tibial nails, and have correspondingly shorter lengths for the long bones of the upper extremities. The body portion of the nails of the present invention have diameters in the range from about 8 to 16 mm and more typically from about 7 to 10 mm for femoral nails and from about 7.5 to 14 mm and more typically from about 8 to 13 mm for tibial nails, and have correspondingly smaller diameters for the long bones of the upper extremities. The proximal end portions or extensions of the nails of the present invention have diameters which range from about 0 to 4 mm and more typically from about 0.5 to 3 mm greater than their corresponding body portions.

The diameters of the bores of the proximal end of the universal bore patterns of the present invention may each have any suitable dimension, and preferably have the same diameter from bore to bore and from universal proximal bore pattern to universal proximal bore pattern to further minimize the number of components to be inventoried. In the illustrated embodiments of FIGS. 1, 2 and 3, the diameters of the proximal bores have been selected to be about 5.5 mm for both the femoral and tibial nails but may be in the range from about 5.0 to 6.5 mm, and more typically from about 5.8 to 6.1 mm. Additionally, the proximal bores may be threaded to correspond with the bone screws.

The nails of the present invention and components or extensions thereof, may be made of biocompatible materials, preferably metals and metal alloys, having a suitable strength and durability to withstand the stresses and strains of their intended function. Suitable materials include but are not limited to surgical grade stainless steel, titanium, vitallium, chrome-cobalt alloys and the like.

The subject nails commonly have a cylindrical cross-sectional shape but may have any suitable shape, including but not limited to oval, elliptical, star-shaped, or triangular with rounded points. The subject nails may also be curved along their lengths, such as in the antero-posterior or medio-lateral directions, and may have longitudinal scalloping as well as various types of tapering or beveling at the proximal and distal ends of the nails to facilitate implantation thereof.

Systems

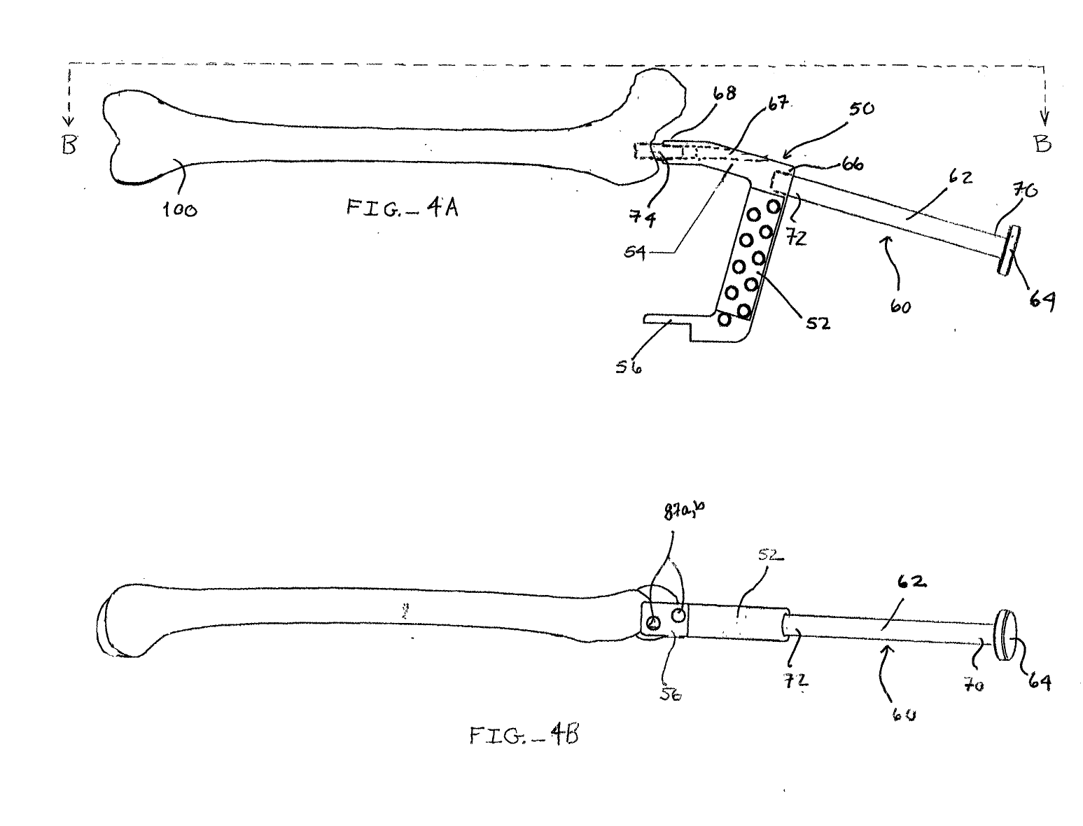

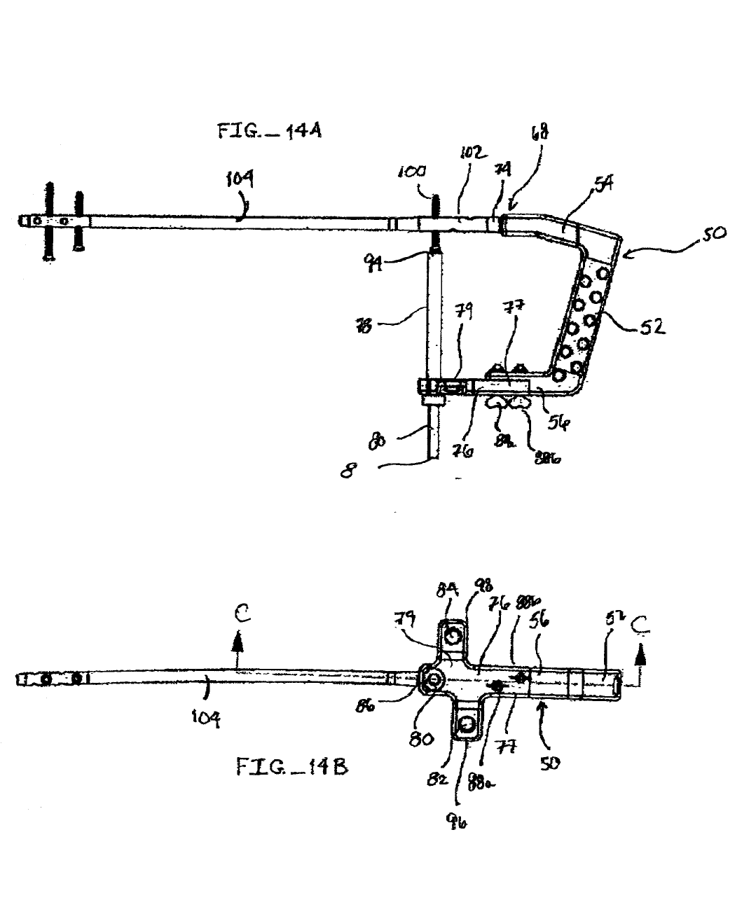

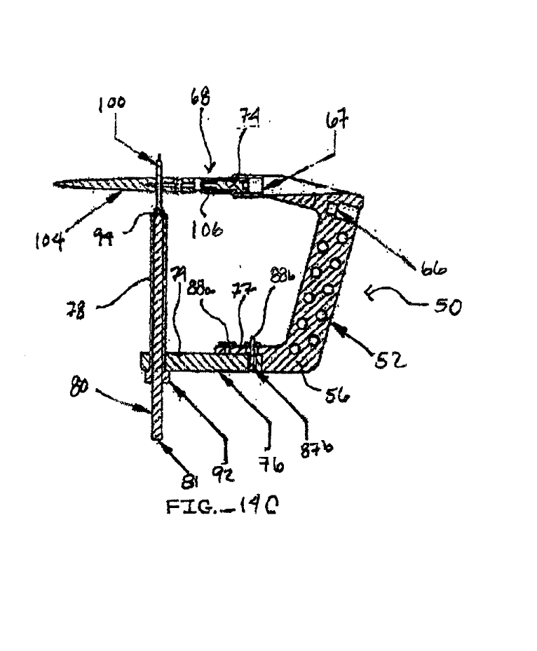

As mentioned above, the present invention also includes systems for implanting the intramedullary nails of the present invention into long bones. Referring now to FIGS. 14A-C, the subject systems include at least a nail insertion and locking assembly 50, as also shown in FIGS. 4A, 4B, 7A, 7B, 10A and 10B, operatively engaged with a proximal end portion 104 of an intramedullary nail 104 for inserting and locking the intramedullary nails of the present invention within the medullary canal of a targeted long bone. The nail insertion and locking assembly 50 includes various components having respective configurations which enable it to be compatible with the intramedullary nails and nail extensions of the present invention. More particularly, the components of assembly 50 which are used to hold and/or position the bone screws for drilling into the universal proximal bores of the subject nails have configurations corresponding to the relative positions, locations and/or sizes of the corresponding universal proximal bores of the intramedullary nails of the present invention.

Generally, nail insertion and locking assembly 50 includes a handle portion 52, a drive portion 54 and a drill guide attachment portion 56. Handle portion 52 primarily allows the surgeon or other surgical personnel to hold and manipulate nail insertion assembly 50 while inserting and locking the intramedullary nails within the bone.

As illustrated in FIGS. 4A, 4B, 7A, 7B, 10A, and 10B, certain systems of the present invention may include a strike plate 60 which is operatively engageable with nail insertion and locking assembly 50, together providing the means for properly inserting and seating a nail within the medullary canal of a long bone. Strike plate 60 includes a hammer shaft 62 and a hammer plate 64 attached to the proximal end 70 of strike plate 60. The proximal end of drive portion 54 of nail insertion assembly 50 has a strike plate interface receptacles 66 and 67. Interface receptacle 66 is the recommended location for insertion of strike plate 60 for antegrade femoral applications, while interface receptacle 67 is the recommended location for insertion of strike plate 60 for retrograde femoral and tibial applications. Both receptacles 66, 67 are configured to receive and engage distal end 72 of hammer shaft 62 of strike plate 60. As best illustrated in FIGS. 14A and 14C, distal end 68 of drive portion 54 provides an attachment pin 74 extending there from and configured for engaging with attachment groove 12 of proximal end portion 6 of the subject intramedullary nails and/or extension thereof.

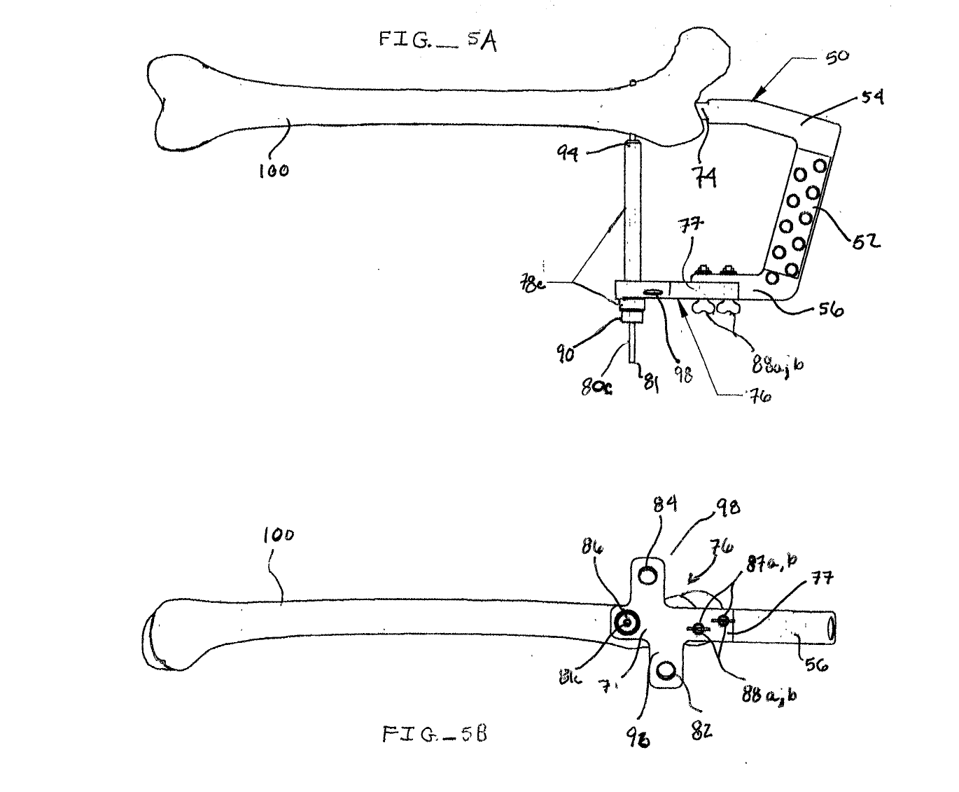

The systems of the present invention include, in addition to nail insertion and locking assembly 50, at least one universal drill guide or jig 76, at least one screw insertion sleeve 78, at least one universal drill bit 80, at least one drill insertion sleeve 90, at least one universal screw drive shaft (e.g., a hexdriver shaft) (not shown) and at least one bone screw, as illustrated in FIGS. 5A, 5B, 8A, 8B, 11A, 11B and 14A-C. Drill guide or jig 76 has a base portion 77 having two screw holes 87a, 87b therein for receiving ¼ turn fasteners 88a, 88b for mounting drill guide 76 to drill guide attachment portion 56 of nail insertion assembly 50. Drill guide or jig 76 has support portion 79 having guide bores 82, 84 and 86, each for receiving and supporting a drill insertion sleeve 78, drill insertion sleeve 90 and a universal drill bit 80. A single drill bit 80 is used to drill all bores within the bone. The proximal end 81 of drill bit 80 is configured for mating engagement with a drill or modular handle (not shown).

The longitudinal axes of each of guide bores 82, 84 and 86 of the universal guide bore pattern of universal drill guide 76 are concentric respectively to corresponding transverse proximal bores 16, 18, and 20 of universal bore pattern 14 of proximal end portion 6. As such, lateral extensions 96 and 98 of drill guide 76, which provide guide bores 82 and 84, respectively, are each offset or extend upwards from drill guide 76 at an angle or angles corresponding to the angle(s) at which proximal bores 16 and 18 are canted, anteverted or retroverted with respect to the longitudinal reference plane and/or the transverse reference plane, as described above. As such, it is necessary to inventory only a single-configuration universal drill guide (and only corresponding single configuration universal drill sleeve, universal screw insertion sleeve, screw insertion drive shaft, drill bit, discussed in more detail below) of the present invention per each universal proximal bore pattern of the intramedullary nails of the present invention.

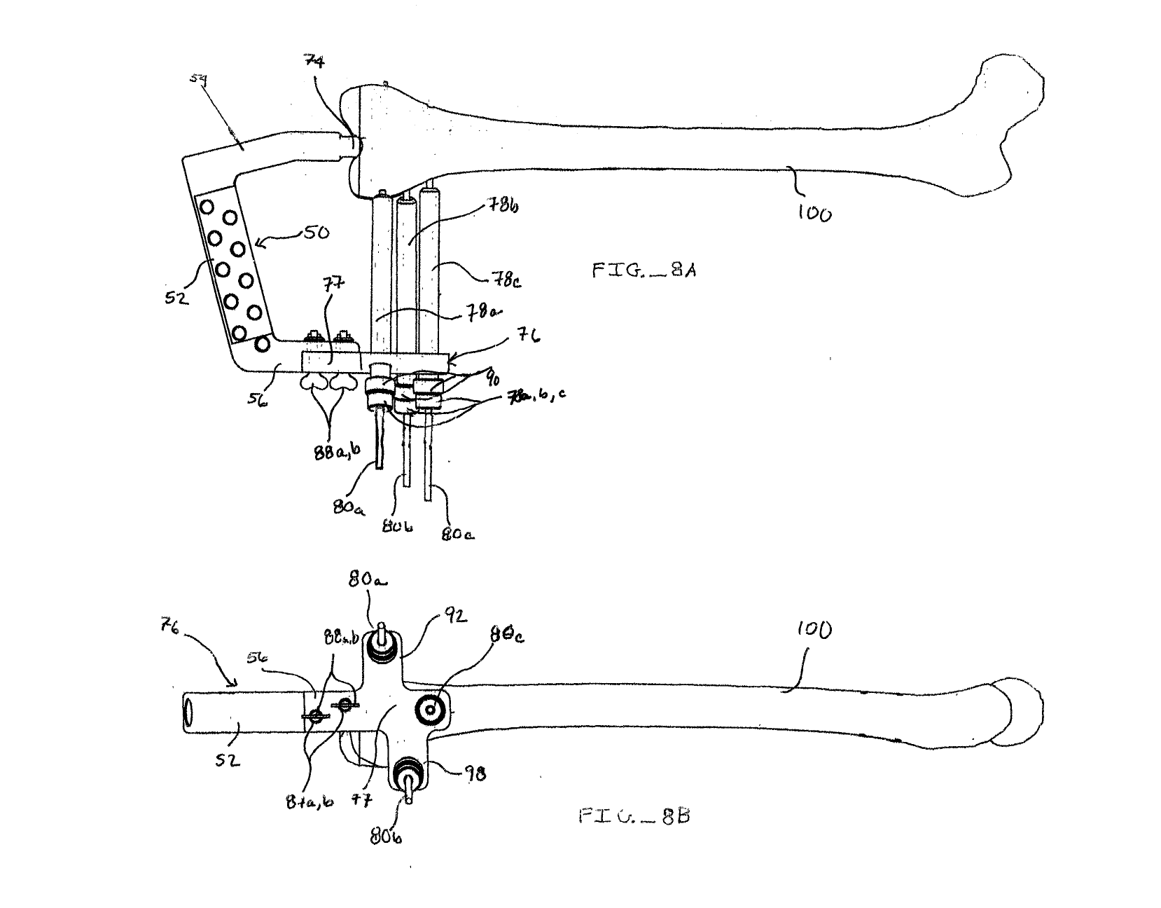

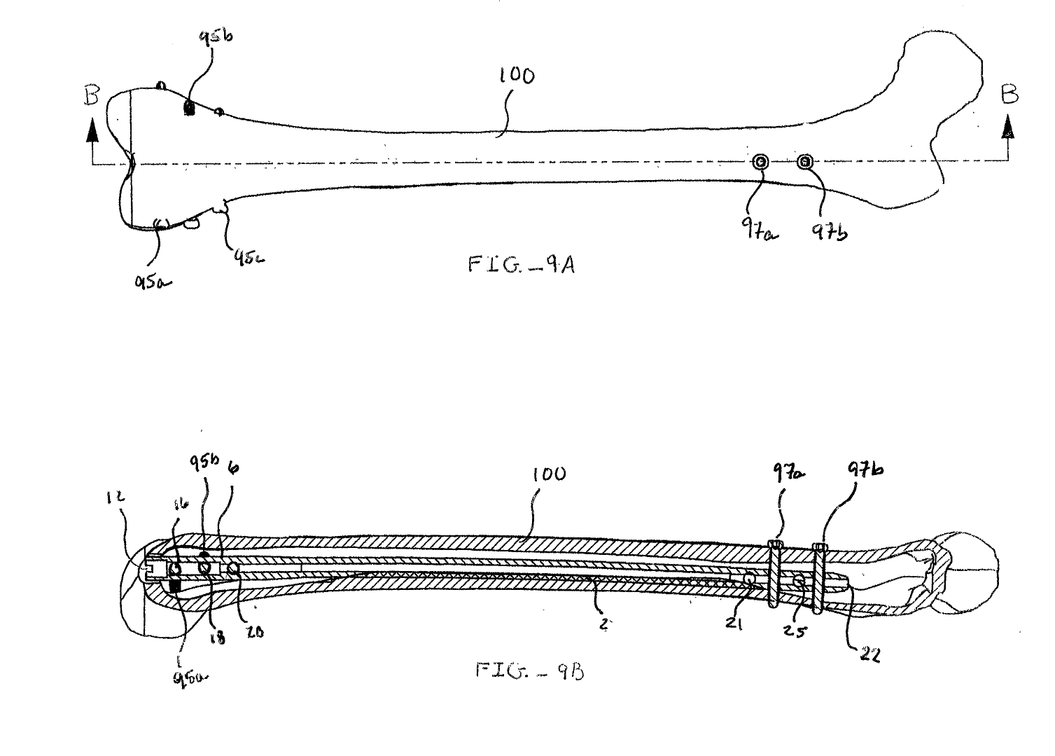

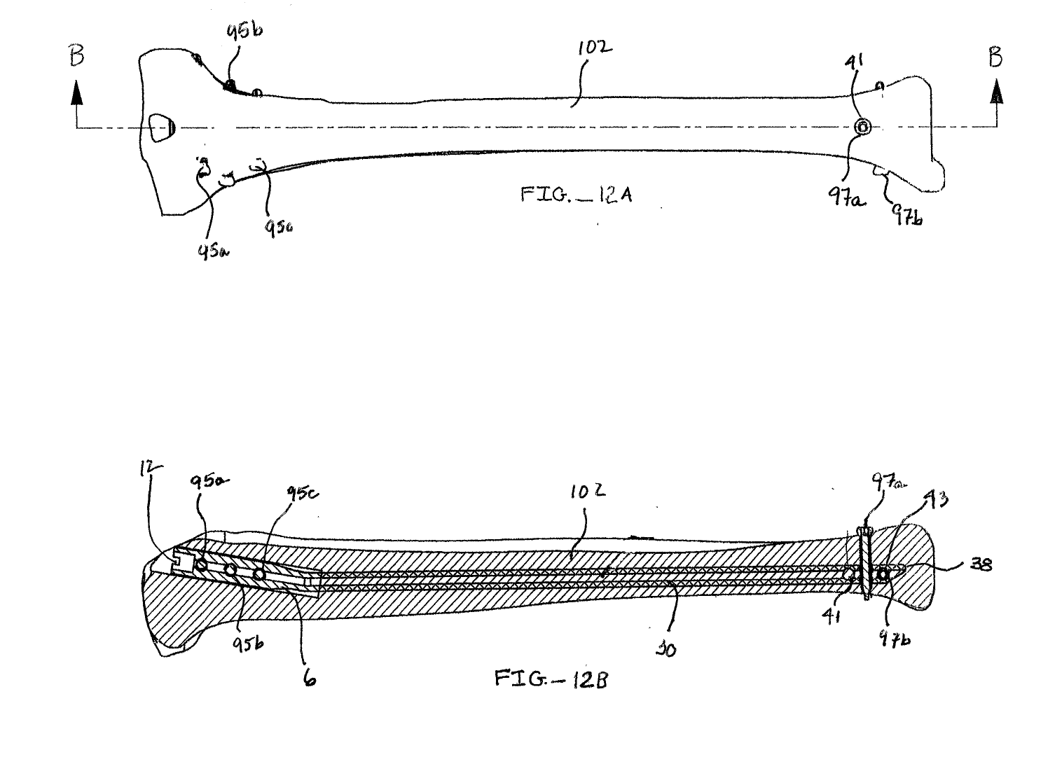

The subject system also includes at least one screw insertion sleeve 78c for stabilizing and guiding a respective at least one drill bit 80c during the drilling process (only one screw insertion sleeve 78c is shown in the embodiment of FIGS. 5A, 5B, and 14A-C while three screw insertion sleeves 78a, 78b and 78c are shown in the embodiments of FIGS. 8A, 8B, 11A and 11B). Best illustrated in FIGS. 14A and 14C, the distal end 94 of each insertion sleeve 78 is configured to receive and hold the distal end of a proximal screw 100.

The present invention may further include other system embodiments which may include all of the described components, or various combinations thereof.

Methods

The specific methods of intramedullary implantation and fixation of the present invention depend on several factors including, but not limited to, the type of long bone on which the procedure is being performed, the approach or mode, i.e., antegrade or retrograde, of inserting and fixing the nail, and possibly the type of fracture or other indication being treated.

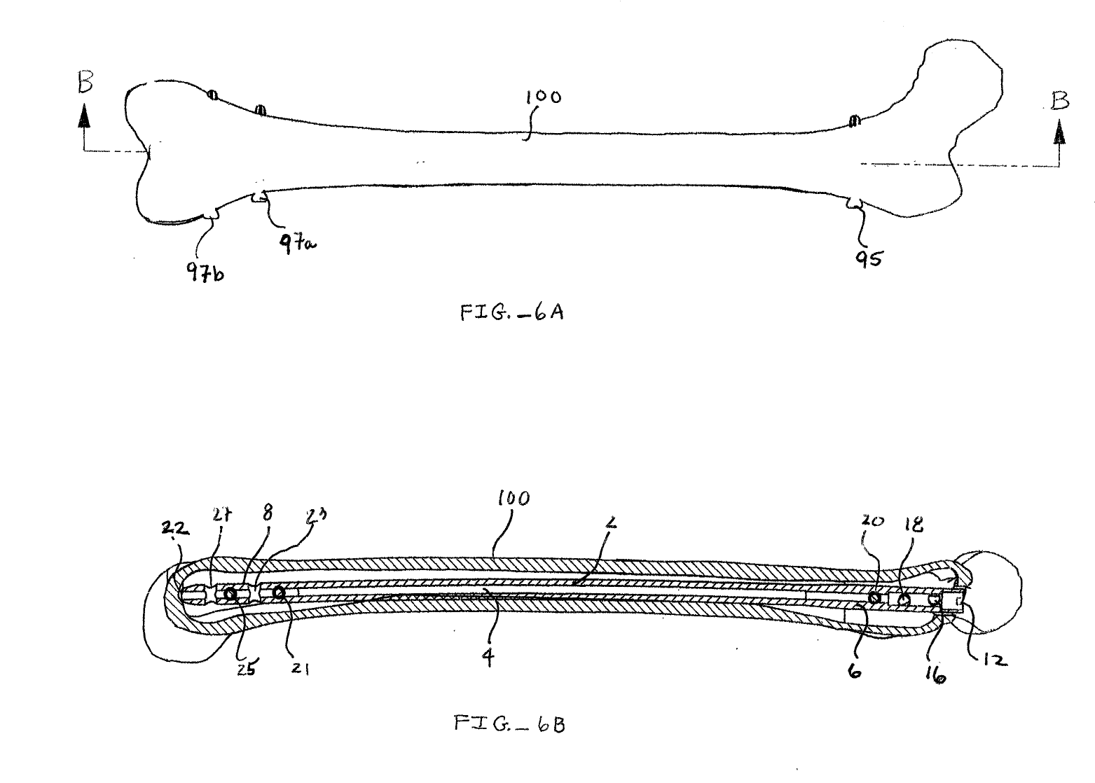

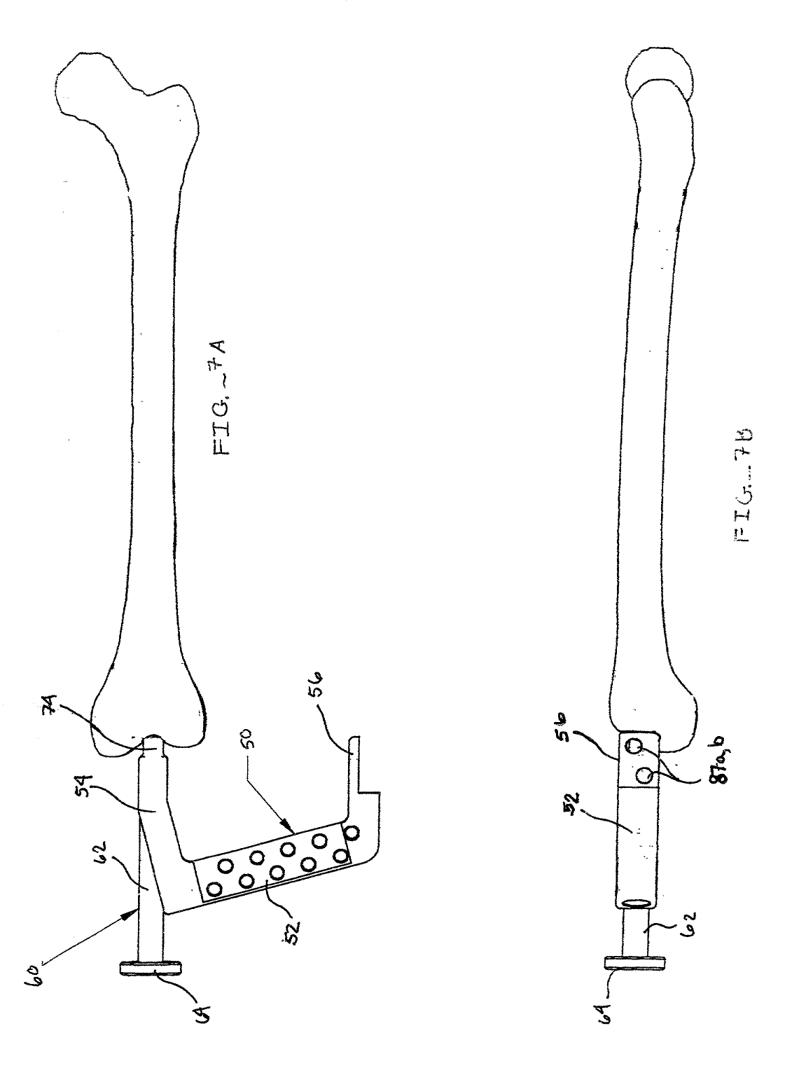

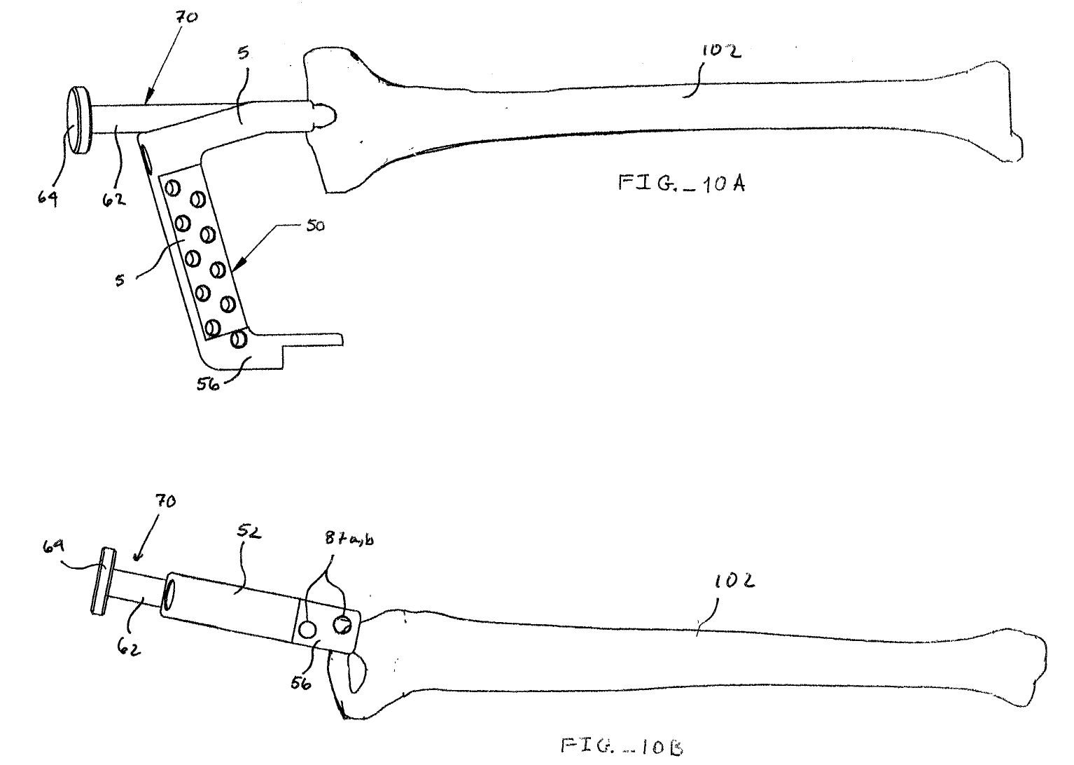

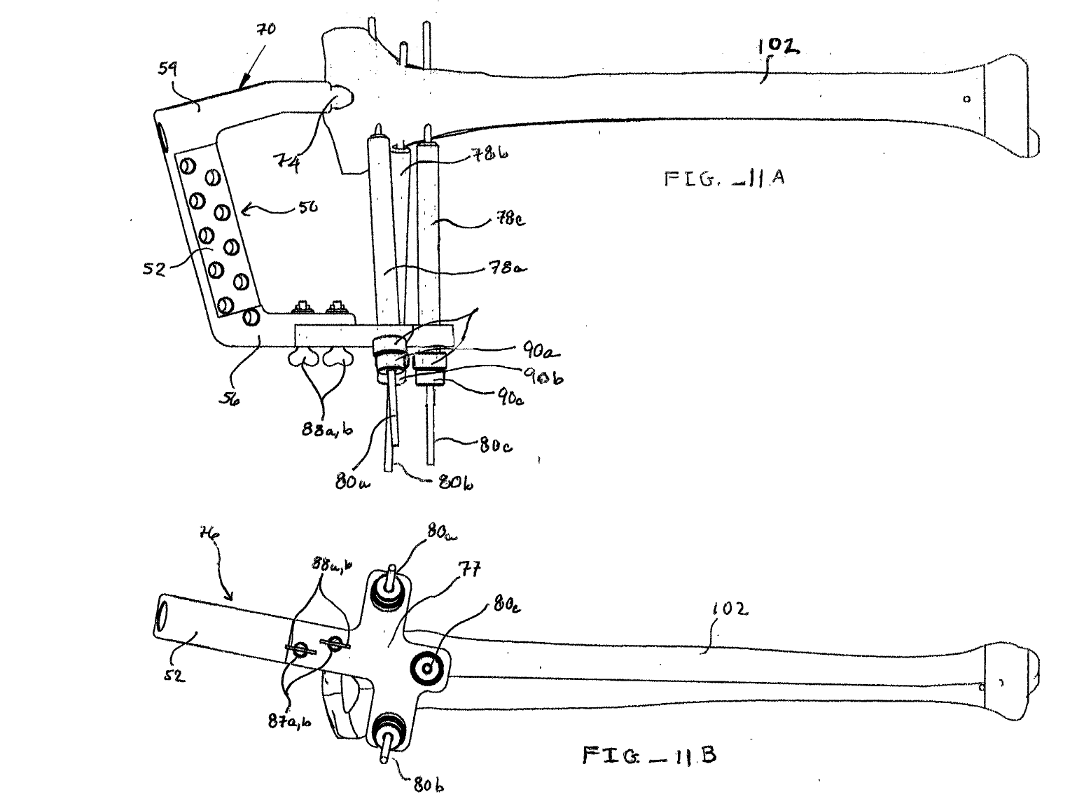

The subject methods will now be generally described with reference to FIGS. 4-12 and to variations in the general procedure as appropriate. In particular, FIGS. 4-6 illustrate the steps for antegrade implantation and fixation of intramedullary nail 2 within a human femur 100, FIGS. 7-9 illustrate the steps for retrograde implantation and fixation of intramedullary nail 2 within a human femur 100, and FIGS. 10-12 illustrate the steps for antegrade implantation and fixation of intramedullary nail 30 within a human tibia 102.

In practicing the subject methods, fluoroscopic imaging may be used as necessary for any of the method steps. Initially, an intramedullary nail of the present invention is provided having a universal bore pattern 14 of the present invention within the parameters discussed above. If the proximal end portion 6 is modular, such a suitable proximal end portion 6 is then selected and operatively attached to the nail's body and distal end portions. In addition to consideration of the factors discussed above, selecting the most suitable nail (or modular nail body portion and distal end portion) also involves determining proper nail length and diameter of the nail for the subject bone(s), such determination being commonly known by those skilled in orthopedic surgery. Additionally, the proper number and diameter of screws must also be determined based on the particular application at hand.

After completing standard patient preparation procedures, an incision is made or an entry port provided to expose the desired section of bone. The site of the incision or entry port will depend on which bone is being treated, the location of the fracture and whether the approach will be antegrade or retrograde. The determination of a suitable incision or entry site is commonly known by those skilled in orthopedic surgery. The nail entry site, the location of which is determined based on whether the surgical approach is antegrade or retrograde, and the intramedullary canal of the subject bone can be prepared by any acceptable manner, such as by reaming or unreamed techniques, commonly known to those skilled in orthopedic surgery.

The properly selected intramedullary nail is then attached to drill handle 52 by means of engaging aligning tabs (not shown) of drill handle 52 with groove 12 of proximal end portion 6 of the nail. An attachment screw (not shown), which is captive to drill handle 52, is then engaged with the threaded portion of the proximal end of the designated nail. The nail is then positioned at the selected entry site of the medullary canal. The distal end 72 of strike plate 60 is then operatively inserted into the proximal end 66 or 67, as appropriate and desired, of drive portion 54 of nail insertion assembly 50. A hammer (not shown) is then used to strike hammer plate 64, advancing the intramedullary nail until optimally positioned within the medullary canal (see FIGS. 4A, 4B, 7A, 7B, 10A and 10B). Strike plate 60 is then unassembled from nail insertion assembly 50.

With drill handle 52 still engaged with the nail which is now operatively positioned within the medullary canal, the universal drill guide 76, having a universal guide bore pattern corresponding to universal proximal bore pattern 14, is then assembled to drill handle 52 as described above. The location and number of fixation points (i.e., screw-bore engagements) along the bone are then determined according to the factors discussed above. Upon a determination of the location of such fixation points, screw insertion sleeve(s) 78 and drill bit insertion sleeve(s) 90 (see FIGS. 5A, 5B, 8A, 8B, 11A and 11B) are operatively assembled and introduced at the appropriate guide bore location (82, 84, or 86) in universal drill guide 76. A calibrated drill bit 80 is passed through the drill insertion sleeve 90 and drilled through the bone and the bores within the universal bore pattern of the nail at the selected fixation point locations to form screw holes therein for receiving bone screws. Calibrated drill bit 80 has graduation marks that prescribe the required bone screw length, so that when the calibrated drill bit forms a hole through both cortices of the bone, the surgeon can extrapolate the required length of the bone screws to be used from the exposed portion of the calibrated drill bit. The drill insertion sleeve 90 is then removed from the screw insertion sleeve 78. A bone screw is then introduced to and operatively engaged with the screw insertion shaft (not shown), which together are inserted into the screw insertion sleeve 90. Screw insertion shaft is then employed to drive the bone screw into the respective screw hole created within the bone. These steps are repeated until a bone screw is implanted at all of the selected fixation points within the bone.

After all the appropriate bone screws have been placed within the proximal end of the nail, securing it within the bone, the drilling and securing of the distal end portion of the nail is then performed by similar means or by means commonly known in the art of orthopedic surgery for drilling bone screws into distal bores of the distal end of an intramedullary nail.

Kits

Also provided by the subject invention are kits for use in practicing the subject methods. The kits of the subject invention include at least one subject intramedullary nail, but oftentimes include a plurality of intramedullary nails. Other kits include at least one subject proximal nail extension of the present invention as well as corresponding nail body and distal end portions. The kits may also include a plurality of proximal and distal transverse screws for locking the intramedullary nails within a bone. Certain kits may further include disposable or reusable instruments and various ancillary components for surgically inserting and fixing the nail within a bone. Such instruments include but are not limited to a nail insertion assembly, a hammer drive, a drill guide, a screw insertion sleeve, a drill bit insertion sleeve, a drill bit, a screw drive or insertion shaft, etc. Such ancillary components include but are not limited to various nuts, screws and pins for assembling the various instruments.

Finally, the kits of the present invention may further include instructions for using the intramedullary nails or extensions thereof, and inserting and fixing them to a bone. The instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., CD-ROM, diskette, etc.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

That which is claimed is:

1. A system for treating long bones, comprising:
at least one intramedullary nail comprising a first universal bore pattern having a plurality of screw bores for receiving screws, each said screw bore defining a longitudinal axis; and
a guide member comprising a second universal bore pattern having a plurality of guide bores for aligning and guiding said screws to be received into said plurality of screw bores, each said guide bore defining a longitudinal axis which is coaxial with a longitudinal axis of a corresponding screw bore.

2. The system of claim 1, wherein the ratio of screw bores to guide bores is 1 to 1.

3. The system of claim 1, wherein said first universal bore pattern is located at a proximal end portion of said intramedullary nail.

4. The system of claim 1, wherein said first universal bore pattern comprises at least three screw bores, from proximally to distally along said intramedullary nail, a first screw bore, a second screw bore and a third screw bore.

5. The system of claim 4, wherein said intramedullary nail defines a longitudinal reference plane wherein the longitudinal axis of each said screw bore is positioned in the range from about negative 60° to about positive 60° with respect to said longitudinal reference plane.

6. The system of claim 5, wherein one of said screw bores is positioned between about 0° to about positive 60° with respect to said longitudinal reference plane.

7. The system of claim 5, wherein another of said screw bores is positioned between about 0° to about negative 60° with respect to said longitudinal reference plane.

8. The system of claim 5, wherein said first screw bore is positioned between about 0° to about positive 60° with respect to said longitudinal reference plane and said second screw bore is positioned between about 0° to about negative 60° with respect to said longitudinal reference plane.

9. The system of claim 8, wherein said first screw bore is positioned between about 0° to about positive 15° with respect to said longitudinal reference plane and said second screw bore is positioned between about 0° to about negative 15° with respect to said longitudinal reference plane.

10. The system of claim 9, wherein said first screw bore is positioned at about positive 15° with respect to said longitudinal reference plane, said second screw bore is positioned at about negative 15° with respect to said longitudinal reference plane, and said third screw bore is positioned at about 0° with respect to said longitudinal reference plane.

11. The system of claim 4, wherein said intramedullary nail defines a transverse reference plane perpendicular to said longitudinal reference plane and wherein the longitudinal axis of each said screw bore is positioned in the range from about negative 60° to about positive 60° with respect to said transverse reference plane.

12. The system of claim 4, wherein said proximal end portion of said intramedullary nail comprises a proximal end and wherein the distance of each said screw bore from said proximal end increases at a fixed increment.

13. The system of claim 12, wherein said fixed increment is about 15 mm.

14. The system of claim 4, wherein said proximal end portion of said intramedullary nail comprises a proximal end and wherein the distance of each said screw bore from said proximal end increases at increments of varying lengths.

15. The system of claim 4 wherein said proximal end portion of said intramedullary nail comprises a proximal end and wherein the distance of each said screw bore from said proximal end increases at an increment in the range from about 8 to 75 mm.

16. A system for treating long bones, comprising:
at least one intramedullary nail configured for treating the femur comprising a plurality of screw bores at a proximal end for receiving screws, said plurality of screw bores defining a selected screw bore pattern; and
at least one intramedullary nail configured for treating the tibia comprising a plurality of screw bores at a proximal end for receiving screws, said plurality of screw bores defining said selected screw bore pattern.

17. The system of claim 16 further comprising a guide member for aligning and guiding interlocking screws to be received into said screw bores of said selected screw bore patterns of said femoral nail and said tibial nail.

18. The system of claim 16 wherein said at least one femoral nail is configured for antegrade and retrograde implantation in a left and right femur and said tibial nail is configured for antegrade implantation in a left and right tibia.

19. The system of claim 16 further comprising a nail insertion and locking assembly comprising a guide member for aligning and guiding interlocking screws to be received into said screw bores of said selected screw bore patterns of said femoral nail and said tibial nail.

20. A method of implanting an intramedullary nail into a long bone, comprising:
providing the system of claim 1;
selecting an appropriate intramedullary nail;
forming a nail entry site at an appropriate location proximate the long bone;
through said entry site, preparing the medullary canal of said long bone for receiving said intramedullary nail;
inserting said intramedullary nail into said prepared medullary canal;
determining which of said screw bores of said intramedullary nail to use as fixation points within the bone;
operatively aligning said guide bores of said guide member with said screw bores of said intramedullary nail, wherein an alignment path is defined between each said guide bore and each said screw bore;
drilling a screw hole within the bone at each said fixation point by means of a drill bit guided along said alignment path; and
delivering a bone screw into each said screw hole.

21. The method of claim 20 wherein said long bone is a femur and said entry site is adjacent to a proximal end of said femur.

22. The method of claim 20 wherein said long bone is a femur and said entry site is adjacent to a distal end of said femur.

23. The method of claim 20 wherein said long bone is a tibia and said entry site is adjacent to a proximal end of said tibia.

24. A kit for treating long bones, comprising a plurality of intramedullary nails having various length and diameter dimensions wherein each said intramedullary nail comprises the same screw bore pattern.

25. The kit of claim 24 wherein said plurality of intramedullary nails comprises nails configured for treating both left and right femurs and nails configured for treating both left and right tibias.

26. The kit of claim 25 wherein each of said femoral nails is configured for implantation by an antegrade approach and a retrograde approach and each of said tibial nails is configured for implantation by an antegrade approach.

27. The kit of claim 24 wherein said screw bore pattern comprises a plurality of screw bores for receiving screws, each said screw bore defining a longitudinal axis, said kit further comprising a guide member comprising a guide bore pattern having a plurality of guide bores for aligning and guiding said screws to be received into said plurality of screw bores, each said guide bore defining a longitudinal axis which is coaxial with a longitudinal axis of a corresponding screw bore.
